(19) United States
(12) Patent Application Publication
(10) Pub. No.: US 2025/0339085 A1
(43) Pub. Date: Nov. 6, 2025

(54) SYSTEM AND METHOD FOR ASSESSMENT OF CHILDREN

(71) Applicant: Gab Technology Corp., Oshawa (CA)

(72) Inventors: Mahtab Mohammadi, Karaj (IR); Mahasta Babaey, Tehran (IR); Amirreza Izadi, Tehran (IR)

(73) Assignee: Gab Technology Corp., Oshawa (CA)

(21) Appl. No.: 18/655,523

(22) Filed: May 6, 2024

(51) Int. Cl. A61B 5/00; G16H 15/00; G16H 20/30

(52) U.S. Cl. CPC A61B 5/40; G16H 15/00; G16H 20/30; A61B 2503/06

(57) ABSTRACT

A system and method of collecting, analyzing, and assessing children's participation data in kindergarten activities is disclosed. The method divides a kindergarten setting into a plurality of zones assigned to a set of activities. Data on children's entrance and exit times for each zone, children's development level, children's interests and preferences are obtained from an RFID-based data collector comprising of one or more RFID tags that are embedded in bracelets or other wearable devices and are worn by one or more individual children, and one or more RFID readers that capture real-time data from the RFID tags associated with individual children, and a server that connects to the RFID readers to store and process data.

### FIELD OF THE INVENTION

The present invention relates to a system and method for collecting and analyzing data about children's participation in different activities in kindergarten, and assessing their development based on various aspects of their growth.

### BACKGROUND OF THE INVENTION

It is crucial for educators, parents, and caregivers to have a deep understanding of the development and character features of children in early childhood education because it directly impacts their future academic, social, and emotional well-being. Kindergartens, being a fundamental component of early education, assume a vital responsibility in fostering and facilitating this development and character features. Therefore, it is essential to observe and assess the progress of children across different developmental domains and different character features within the kindergarten setting.

However, the current methods used to assess children in kindergarten frequently encounter issues of subjectivity, inconsistency, and incompleteness. They lack the required objectivity and comprehensiveness to offer a thorough understanding of a child's overall development and character features. For example, some of these approaches heavily rely on teachers' observations or reports, which can vary depending on individual teacher experiences, biases, or workload. Standardized tests or surveys, although utilized in certain cases, may not encompass the entire range of children's abilities, interests, or personalities.

While these methods do provide valuable insights, they have several shortcomings. One drawback is the absence of timely, continuous, and personalized feedback for teachers, parents, and the children themselves. This lack prevents parents from receiving ongoing information necessary for effective support and intervention. These methods often fail to reflect the dynamic and diverse nature of children's learning experiences in kindergarten. Instead, they tend to concentrate on isolated skills or outcomes, disregarding the significance of holistic and integrated development in children.

To address these issues, the present invention introduces a method and system that assesses children based on their participation in different activities. Advancements in technology, particularly in the field of wireless communication and automatic identification, have paved the way for innovative approaches to assess and analyze children's development in educational environments. The use of wireless communication technology to document children's activities has emerged as a promising method to gather objective and reliable data. By leveraging this technology, it becomes possible to collect detailed information about the timing, frequency, and duration of each child's participation in activities within a kindergarten setting.

The present invention relates to a system and method designed to evaluate the development of children in kindergarten through their participation in different activities covering various subjects and developmental aspects. This system and method divide the kindergarten into a plurality of zones and assign activities to each zone. To track the time, duration, and frequency of each child engaging in each zone, the system and method utilize multiple data collectors based on RFID technology. In the implementation, each zone is equipped with one or more RFID readers, and each child is provided with an RFID tag that is activated by the RFID readers. By analyzing the time data collected and employing algorithms, the system and method of this invention detect patterns in children's behavior and assess their level of participation in the activities associated with each zone.

The assessment conducted by this invention encompasses several aspects, including children's development, interests, preferences, time of peak engagement, attention spans, napping habits, as well as social behaviors and interactions. By considering these factors, the assessment offers a comprehensive overview of each child's attributes and requirements concerning their learning and development.

The invention generates a report that incorporates the data gathered during the assessment. This report provides guidance, feedback, and insights regarding the child's growth and development, as well as highlighting any potential issues that may need attention.

### SUMMARY OF THE INVENTION

A system and method of collecting, analyzing, and assessing children's participation data in kindergarten activities is disclosed. The method divides a kindergarten setting into a plurality of zones. Each of the plurality of zones is assigned to a set of activities. Data on children's entrance and exit times for each zone is obtained from an RFID-based data collector. Zone attendance data that indicates the time, frequency, and duration of each child's presence within each zone is generated by analyzing data obtained from the RFID-based data collector. By interpreting wherein children's presence within each zone as their participation in the set of activities assigned to the respective zone, participation data for each child that indicates the time, frequency, duration, and sequence of the respective child's participation in the set of activities assigned to each zone is generated using zone attendance data. Then, children are assessed based on participation data and a plurality of factors related to children's characteristics comprising but not limited to their development level, interests and preferences, time of peak engagement, duration of attention, napping patterns, and social behaviors and interactions and potential strengths and problems in children's characteristics are identified. A report on the plurality of factors related to children's characteristics and participation data is then generated.

Children's development level is identified across a plurality of development criteria that are aligned with key aspects of child growth and development. Children's interests and preferences are assessed across a plurality of interest criteria. The kindergarten setting is optimized to enhance children's learning and overall development. To optimize the kindergarten setting a participation percentage for each of the plurality of zones and each of the plurality of subdomains is obtained. Each of the plurality of the development criteria is assigned to one of the development stages comprising developed criteria, underdeveloped criteria, and overdeveloped criteria. To maintain the current participation percentage for zones and subdomains that influence the developed criteria, while improving the participation percentage for zones and subdomains that impact the underdeveloped criteria, a plurality of redesign suggestions is identified. The plurality of redesign suggestions comprises rearranging the positions of zones, assigning a subdomain to its respective zone for a shorter duration, and assigning a subdomain to its respective zone for a longer duration. A report is then generated capturing the result of assessing children in the set of aspects and the redesign suggestions for the kindergarten setting.

The present invention utilizes an RFID-based data collector to identify one or more individual child and their position. The RFID-based data collector comprises one or more RFID tags that are embedded in bracelets or other wearable devices and are worn by one or more individual children, one or more RFID readers that capture real-time data from the RFID tags associated with individual children, and server that connects to the RFID readers to store and process data. A plurality of activity assignment modules respectively placed in kindergarten wherein it divides the kindergarten into a plurality of zones and each zone is assigned to a set of activities. Each zone is equipped with one or more RFID readers. A plurality of data analysis modules is used to apply a plurality of algorithms to data collected from the RFID-based data collector. The report generation module generates reports of assessment based on the data analysis modules' results and wherein enables the user to modify the report according to their preferences.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 shows a zone and the equipment used in a zone;

FIG. 2 shows a kindergarten divided into a plurality of zones and the equipment used in the kindergarten;

FIG. 3 shows a flowchart of one embodiment of the method for assessment of children;

FIG. 4 shows the distribution of time spent by a child (User 1) across different zones and subdomains through a pie chart;

FIG. 5 shows the participation value of a child (User 2) in interest criteria through a spider chart;

FIG. 6 shows the participation value of a child (User 3) in development criteria through a spider chart;

FIG. 7 shows the impact values of zones and subdomains on the interest criteria through a snaky chart

FIG. 8 shows a social interaction map for a child (User 1);

FIG. 9 shows a dynamic characteristic map created for a child (user 1);

FIG. 10 shows a report of the optimizing kindergarten setting.

### DETAILED DESCRIPTION OF THE INVENTION

The present invention comprises a system and a method for assessment of children based on their participation in activities within a kindergarten setting. The system comprises a plurality of activity assignment modules that are used to divide kindergarten into a plurality of zones and assign a set of activities to each zone. In this context, zones represent designated areas that focus on distinct aspects of early childhood education and development. The set of activities assigned to each zone is aligned with the corresponding aspect of early childhood education and development addressed within that zone.

In one embodiment of the method, a rotation schedule is defined for the set of activities within each zone. To assign the set of activities to each zone, a plurality of main domains that cover a broad area of early childhood education and development is defined. Each main domain includes a plurality of subdomains, and each subdomain includes a plurality of activities. The subdomains break down each main domain into more specific areas and the activities in each subdomain are designed to address the specific learning objectives of the subdomain. For one embodiment of the present invention, the main domains, the subdomains, and the sets of activities are listed in Table 1. Each main domain is assigned to one of the plurality of the zones. This means that each zone is dedicated to a specific subject area. Within each zone, one of the plurality of subdomains is chosen and assigned for a designated time which can be one or more days. During this designated time, activities associated with the respective subdomain are provided for children in that zone. After the designated time, a different subdomain is selected, and its activities are provided within the zone. For instance, science and technology are subdomains of the STEM zone. Activities included in the science subdomain can be provided in the STEM zone for 2 days, and then activities included in the technology subdomain can be provided in the STEM zone for 1 day.

TABLE 1

| Main Domains | Subdomains | activities |
|---|---|---|
| STEM | Science | Mathematics |
| | | Physics |
| | | Biology |
| | | Space |
| | | Chemistry |
| | Technology | Electronics |
| | | Robotics |
| | | Computer |
| Learning | Library | Fiction book |
| | | Non-fiction book |
| | Learning Games | Puzzles |
| | | Memory matching games |
| | | Word games |
| | Board Games | Teamwork Board Games |
| | | Strategic Board Games |
| | | Thinking Board Games |
| | | Problem-solving Board Games |
| Physical Activity | Gross Motor Skills | Running and Jumping |
| | | Climbing |
| | | Crawling |
| | | Playing with balls |
| | | Playing with . . . |
| | Fine Motor Skill | Drawing |
| | | Painting |
| | | Cutting |
| | | Threading beads |
| | | Playing with puzzles |
| | | Manipulating small objects |
| Outdoor Activity | Zoology | Animal-themed play structures |
| | | Animal replicas |
| | Gardening | Planting seeds |
| | | Watering plants |
| | | Weeding |
| | | Harvesting |
| | Sandbox | Building sandcastles |
| | | Digging |
| | | Molding shapes with sand |
| Art and Creation | Music | Play Drums |
| | | Play Xylophones |
| | | Play Keyboards |
| | | Play audio devices |
| | Painting | Easels |
| | | Paints |
| | | Brushes |
| | | Pencils |
| | | Brushes |
| | | Paper |
| | | Watercolor |
| | | Crayon |
| | | Fingerpaint |
| | Role-playing | History events |
| | | Kitchen |
| | | Doctor's office |
| | | Grocery store |
| | Sculpture | Clay |
| | | Modeling dough |
| | | Wire |
| | Craft | Collages |
| | | Paper crafts |
| | | Origamis |

The invention uses an RFID-based data collector to identify children's positions and obtain real-time data on the times that each child enters and exits each of the zones. The RFID-based data collector comprises a plurality of RFID tags, a plurality of RFID readers, and a server. Each RFID tag has a code that serves as a unique identifier. Each RFID reader has a read range area and is able to identify RFID tags within its read range area and sends a signal containing the RFID tag's code to the server. The server stores and processes data received from the RFID readers.

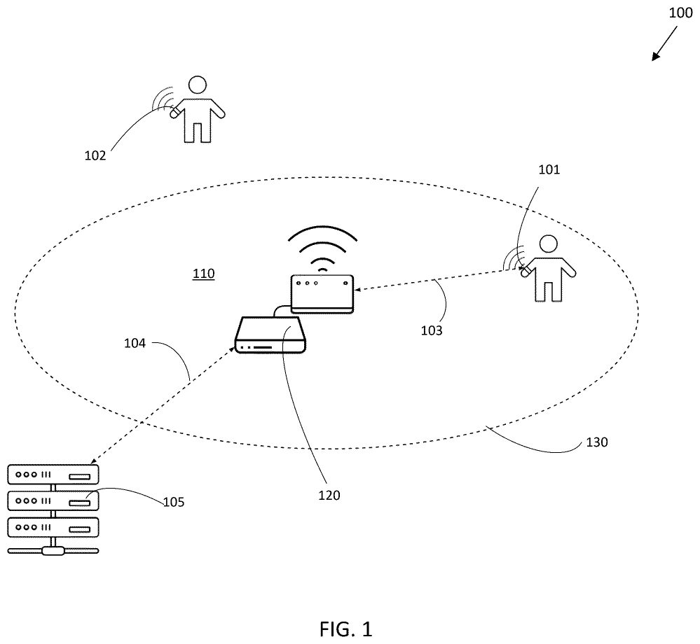

FIG. 1 illustrates the equipment used in each zone to obtain real-time data on the times that each child enters and exits each of the zones. The zone 110 is equipped with an RFID reader 120. Each of the individual children wears an RFID tag 101 and 102 that can be attached to a bracelet or a wearable device. Each RFID tag 101 and 102 has a unique identifier in the form of a code that corresponds to the child's identity. The RFID reader 120 can send and receive certain signals to identify RFID tags 101 and 102 in a certain area called the read range area 130. When a child equipped with an RFID tag 101 enters the read range area of the RFID reader 130, the RFID reader's signal 103 triggers the RFID tag 101 to send its code back to the RFID reader 120. When a child equipped with an RFID tag 102 leaves the read range area of the RFID reader 130, the RFID reader's signal does not reach the RFID tag 102 and the RFID tag 102 does not send its code back to the RFID reader 120. The RFID tag 101 stays active as long as the child is in the read range area of the RFID reader 130, which allows the RFID reader 120 to determine the exact time of entry and exit for each child equipped with an RFID tag. The RFID reader 120 then sends this time data to server 105 via network 104. The server 105 is configured to store and analyze the time data received from the RFID reader 120.

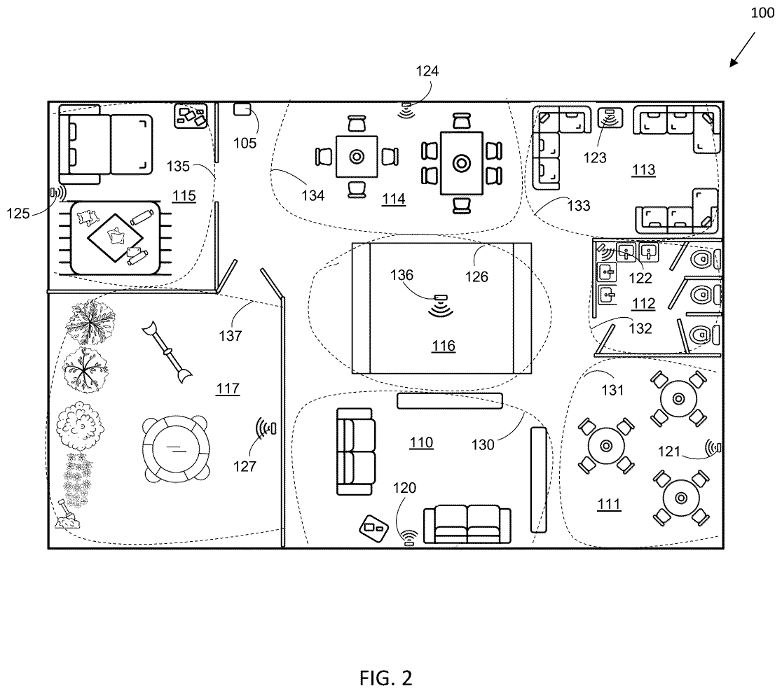

FIG. 2 illustrates the equipment used in a kindergarten 100 to obtain data on the times that each child enters and exits each of the zones. Kindergarten 100 is divided into a plurality of zones 110-117 and each zone is equipped with one or more RFID readers 120-127 that trigger RFID tags worn by children when they enter the zones 110-117. The zones' borders are determined by the read range area of the RFID readers 130-137. When a child equipped with an RFID tag passes the read range area of an RFID reader 130-137 and enters the zone 110-117, the RFID reader 120-127 triggers the respective RFID tag to send its code back to the RFID reader 120-127. The RFID reader 120-127, then sends a signal to server 105 to determine the time of activation. When a child equipped with an RFID tag passes the read range area of an RFID reader 130-137 and exits the zone 110-117, the RFID reader 120-127 deactivates the respective RFID tag and stops sending a signal to the server 105. The server 105 is configured to store signals received from RFID readers 120-127 and data of the assignment modules, including dividing kindergarten, activity assignment, and assigning the RFID readers with zones.

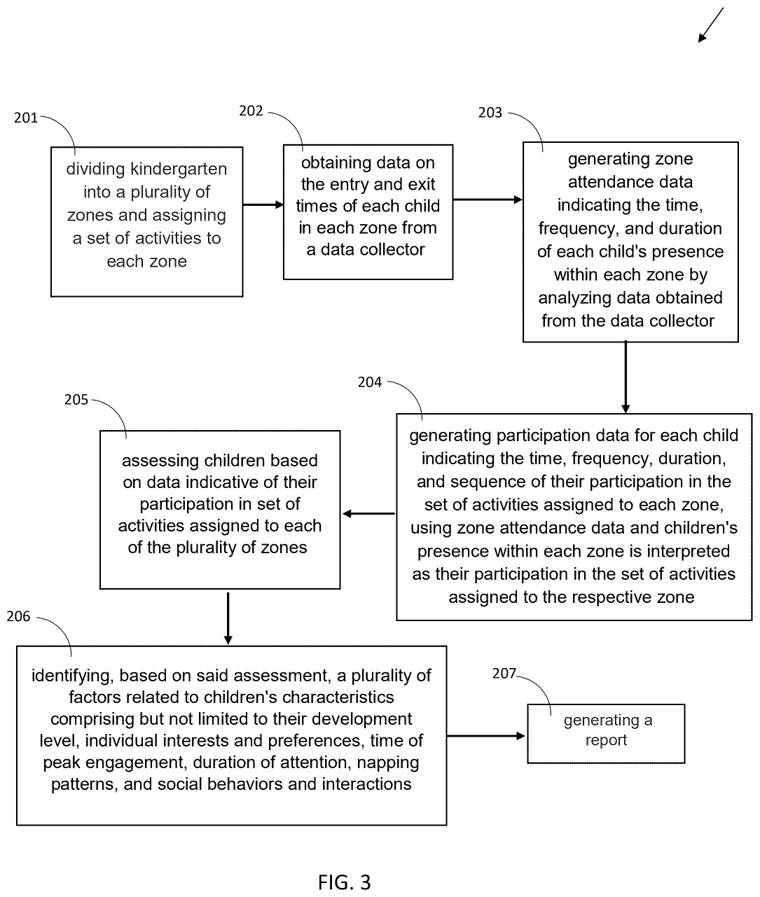

FIG. 3 illustrates one embodiment of the method 200 for assessment of children comprises the steps of dividing kindergarten into a plurality of zones and assigning a set of activities to each zone 201, obtaining data on the entry and exit times of each child in each zone from a data collector 202, generating zone attendance data indicating the time, frequency, and duration of each child's presence within each zone by analyzing data obtained from the data collector 203, generating participation data for each child indicating the time, frequency, duration, and sequence of their participation in the set of activities assigned to each zone, using zone attendance data, and children's presence within each zone is interpreted as their participation in the set of activities assigned to the respective zone 204, assessing children based on data indicative of their participation in the set of activities assigned to each of the plurality of zones 205, identifying, based on said assessment, a plurality of factors related to children's characteristics comprising but not limited to their development level, individual interests and preferences, time of peak engagement, duration of attention, napping patterns, and social behaviors and interactions 206, and generating reports on the plurality of factors related to children's characteristics and on participation data 207.

The system comprises a plurality of data analysis modules that apply a plurality of algorithms to data collected by RFID-based data collection. The data analysis modules analyze the location and time data obtained from the RFID readers and assignment modules to determine the zone in which each of the individual children is engaged. These modules generate zone attendance data that indicates the time, frequency, and duration of each child's presence within each zone. The data analysis modules use this data to generate participation data for each child that indicates the time, frequency, duration, and sequence of their participation in the set of activities assigned to each zone. In this context, children's presence in each zone is interpreted as their participation in the activities assigned to the respective zone.

The invention assesses children based on data that indicates their participation in the set of activities assigned to each of the plurality of zones and identifies a plurality of factors related to children's characteristics comprising but not limited to their development level, interests and preferences, time of peak engagement, total duration of attention, napping patterns, and social behaviors and interactions.

The method identifies children's development levels. In the first step, the method obtains a plurality of development criteria, which are designed to align with key aspects of child growth and development. Each child's development level is identified by measuring the cumulative time they spend engaging in the set of activities that influence these criteria. In the second step, the method determines the impact that the set of activities associated with each subdomain in each zone has on each criterion. In the third step, a plurality of impact values is assigned to each set of activities. Each subdomain or the set of activities associated with each subdomain in each zone can have three levels of impact on each of the development criteria comprising a high impact with an impact value of 5, a medium impact with an impact value of 3, and a low impact with an impact value of 1. The subdomains along with their corresponding impact on the development criteria are detailed in Table 2. In the fourth step, a participation value of each child in each criterion is established. The participation value of each child in each criterion determines the level of the child's participation in relation to each criterion. To establish the participation value of a child in a criterion, the method multiplies the impact value that subdomains have on the respective criterion by the time the child spends in each subdomain relevant to that impact value. The results of these multiplications are then summed together to determine the participation value of the child in the respective criterion. The fifth step is to obtain a participation value requirement for individual children in each criterion. The participation value requirement varies from one child to another and it changes as the child grows and develops. The sixth step is to categorize children's development in each criterion based on the participation value requirement. Children are categorized into three stages, comprising underdeveloped criterion, developed c criterion, and overdeveloped criterion. The underdeveloped criterion implies that the participation value of the child in the respective criterion is lower than its participation value requirement, the developed criterion implies that the participation value of the child in the respective criterion is equal to its participation value requirement, and the overdeveloped criterion implies that participation value of the child in the respective criterion is higher than its participation value requirement.

TABLE 2

| | | Development Criteria | | | | | |
|---|---|---|---|---|---|---|---|
| | | | | Social | Language and | Moral and | Sensory and |
| | | Physical | Cognitive | and Emotional | Communication | Ethical | Perceptual |
| Zones | Subdomains | Development | Development | Development | Development | Development | Development |
| STEM | Science | Low | High | Medium | Medium | Medium | Medium |
| | Technology | Low | High | Medium | Medium | Low | Medium |
| Learning | Library | Low | High | Medium | High | Medium | Medium |
| | Learning games | Low | High | Medium | High | Low | Medium |
| | Board games | Low | High | High | High | Medium | Medium |
| Physical | Gross motor skills | High | Medium | High | Low | Medium | Medium |
| Activity | Fine motor skills | High | Medium | Medium | Low | Medium | Medium |
| Outdoor | Zoology | Low | High | Medium | Medium | Medium | High |
| Activity | Gardening | High | High | High | Medium | Medium | High |
| | Sand box | High | Medium | High | Low | Medium | High |
| Art and | Music | Low | High | High | Medium | Medium | High |
| Creation | Painting | Low | High | High | Medium | Medium | High |
| | Role playing | Low | High | High | High | High | Medium |
| | Sculpture | Low | High | High | Medium | Medium | High |
| | Craft | Low | High | High | Medium | Medium | High |

The method identifies children's interests and preferences. A mandatory participation time for children in kindergarten is obtained which refers to the time during which children are required to participate in activities as directed by the kindergarten teacher, and cannot choose activities themselves. The mandatory participation time is obtained through a user interface by the kindergarten teacher or other caregivers, and participation data excluding the mandatory participation time are generated. Through the analysis of children's participation data without mandatory time, the total amount of time each child spends in each zone and subdomain is determined. The zones and subdomains, with the highest participation time, are identified as children's interests and activity preferences.

The method also identifies children's interests and preferences within a plurality of interest criteria. The plurality of interest criteria which encapsulate a broad spectrum of children's interests, are obtained. The method determines the impact that each subdomain has on each criterion. Each subdomain can have three levels of impact on each of the interests' criteria comprising a high impact with an impact value of 5, a medium impact with an impact value of 3, and a low impact with an impact value of 1. The subdomains along with their corresponding impact on the development criteria are detailed in Table 3. The cumulative time they spend participating in activities that influence these interest criteria is measured. A participation value for each child in each interest criterion is established wherein the impact value of the corresponding interest criterion is multiplied by the time spent by the child in the relevant subdomain. The results of these multiplications are then summed to determine the participation value of each child in each interest criterion. The interest criterion with the highest participation value indicates the child's interests and preferences.

TABLE 3

| | | Interest Criteria | | | | | | |
|---|---|---|---|---|---|---|---|---|
| | | Physical | Pretend | Sensorial | Language and | Artistic | Social | Curiosity and |
| Zones | Subdomains | Play | Play | Exploration | Literacy | Expression | Interaction | Learning |
| STEM | Science | Medium | Low | High | Medium | Low | Medium | High |
| | Technology | Low | Medium | Medium | Medium | Medium | High | High |
| Learning | Library | Low | High | Medium | High | Low | Medium | High |
| | Learning games | Medium | Medium | Medium | High | Low | Medium | Medium |
| | Board games | Low | High | Medium | High | Low | High | Medium |
| Physical | Gross motor skills | High | Medium | Medium | Low | Low | Medium | Medium |
| Activity | Fine motor skills | Low | Medium | Medium | Low | Medium | Low | Medium |
| Outdoor | Zoology | Medium | High | High | Medium | Medium | Medium | High |
| Activity | Gardening | High | High | High | Medium | High | Medium | High |
| | Sand box | High | High | High | Low | Medium | Medium | Medium |
| Art and | Music | Low | High | High | Medium | High | High | High |
| Creation | Painting | Low | High | High | Medium | High | Medium | High |
| | Role playing | Medium | High | Medium | High | High | High | High |
| | Sculpture | Low | High | High | Medium | High | Medium | High |
| | Craft | Low | High | High | Medium | High | High | High |

The time of peak engagement for each child is identified by analyzing their participation data. The time spent by children in kindergarten is divided into smaller segments, comprising morning, noon, afternoon, and periods before or after nap. The frequency and duration of each child's participation in each zone during these time segments are obtained. By examining the data, the time segment with the least frequency of zone changes and the longest duration of participation is identified. This particular time segment indicates when the child is most focused on the set of activities within a zone and less likely to become bored or distracted. It signifies the time of peak engagement for that particular child.

The total duration of attention for each child is identified by analyzing their participation data within different zones. When a child enters a zone and subsequently exits it, the duration of their participation in that particular set of activities is recorded. The number of times the child enters each zone and the duration spent at each entrance are computed. To calculate the duration of attention for a child in each zone, the total duration of the child's presence in the zone is divided by the number of times they enter that specific zone. To identify the total duration of attention for each child, the duration of attention for each child in each zone is divided by the total number of zones.

The total duration of attention for each child during the child's time of peak engagement is also identified by considering only the times when they enter each zone and the duration spent at each entrance during their time of peak engagement. Similar to before, the duration of attention for a child in each zone during their time of peak engagement is calculated by dividing the total duration of their presence in the zone by the number of times they enter that zone specifically during their time of peak engagement. The total duration of attention for each child during their time of peak engagement is then identified by dividing the duration of attention for each child in each zone during their time of peak engagement by the total number of zones.

The method identifies potential problems and strengths in children's total duration of attention. The method establishes a range of age-appropriate duration of attention for each child and categorizes them into three groups by comparing their total duration of attention with their range of age-appropriate duration of attention, comprising children with total duration of attention shorter than their age-appropriate range, children with total duration of attention longer than their age-appropriate range, and children with total duration of attention within their age-appropriate range. A child with a total duration of attention longer than their age-appropriate range might have a talent for staying concentrated. This could indicate a greater chance of success in activities that require focus. A child with a total duration of attention shorter than their age-appropriate range may have underlying issues that prohibit their ability to concentrate and maintain focus. The method identifies possible reasons that could contribute to a shorter duration of attention, including developmental delays, learning disabilities (e.g., ADHD and dyslexia), sensory issues, and underlying medical conditions (e.g., anxiety, depression, or sleep disorders).

The method identifies napping patterns for each child using their participation data. To identify the napping pattern for each child, a zone should be assigned for the nap. The time and duration that each child spends napping in kindergarten is obtained from their participation data. By using this data, the specific timing and durations of children's naps, whether their nap times are consistent or inconsistent, and whether their nap durations remain similar or vary across different napping sessions are determined.

The method identifies potential problems and strengths in children's napping patterns. The method analyzes the post-nap participation level of each child and identifies those with high and low participation levels. To do that, the system identifies each child's duration of attention before and after napping. The total post-nap duration of attention for each child is identified by considering only the times when they enter each zone and the duration spent at each entrance after their nap time. The post-nap duration of attention for a child in each zone is calculated by dividing the total duration of their presence in the zone by the number of times they enter that zone, after their nap time. The total post-nap duration of attention for each child is then identified by dividing the post-nap duration of attention for each child in each zone by the total number of zones. The total pre-nap duration of attention for each child is identified similarly to the total post-nap duration of attention. By comparing each child's total post-nap duration of attention to their total pre-nap duration of attention and total duration of attention, the method determines whether they exhibit high or low post-nap participation. Children with high post-nap participation exhibit a longer total post-nap duration of attention compared to their total pre-nap duration of attention and total duration of attention. Children with low post-nap participation exhibit a shorter total post-nap duration of attention compared to their total pre-nap duration of attention and total duration of attention. This method classifies children into eight distinct groups based on the consistency of their nap times, the duration of their naps, and their level of participation after napping. These categories comprise children with consistent nap times of similar duration and high post-nap participation, children with consistent nap times of variable duration and high post-nap participation, children with inconsistent nap times of similar duration and high post-nap participation, children with inconsistent nap times of variable duration and high post-nap participation, children with consistent nap times of similar duration and low post-nap participation, children with consistent nap times of variable duration and low post-nap participation, children with inconsistent nap times of similar duration and low post-nap participation, and children with inconsistent nap times of variable duration and low post-nap participation. The method identifies a plurality of potential problems that may contribute to low post-nap participation in groups of children and offers a corresponding set of possible solutions for each identified problem. In one embodiment of the method, the potential issues and their associated solutions for children with low post-nap participation are detailed in Table 4.

TABLE 4
the Potential Issues and their Associated Solutions

| Group of Children | Potential Issues | Solutions |
|---|---|---|
| Children with consistent nap | Napps are too long | Shortening the child's nap |
| times of similar duration and | Napps are too short | Extending the child's nap |
| low post-nap participation | Napping time is too late in | Adjusting the child's nap time |
| | the day | to earlier in the day |
| | Child's sleep is not deep | Creating a relaxing nap |
| | enough | environment |
| | | Avoiding stimulating activities |
| | | before bed |
| | Chad may have an | Seeing a therapist |
| | underlying sleep disorder | |
| Children with consistent nap | Napps are too long or too | Regulating duration of the |
| times of variable duration and | short | child's nap |
| low post-nap participation | Napping time is too late in | Adjusting the child's nap time |
| | the day | to earlier in the day |
| | Child's sleep is not deep | Creating a relaxing |
| | enough | environment |
| | | Avoiding stimulating activities |
| | | before bed |
| | Child may have an | Seeing a therapist |
| | underlying sleep disorder | |
| | Child may be experiencing | Seeing a therapist |
| | stress or anxiety | |
| Children with inconsistent nap | Child may have sleep | Establish a consistent naptime |
| times of similar duration and | fragmentation | routine |
| low post-nap participation | | Creating a relaxing nap |
| | | environment |
| | | Encourage physical activity |
| | | Avoiding stimulating activities |
| | | before bed |
| | Napps are too long | Shortening the child's nap |
| | Napps are too short | Extending the child's nap |
| | Child may have an | Seeing a therapist |
| | underlying sleep disorder | |
| | Child may be experiencing | Seeing a therapist |
| | stress or anxiety | |
| Children with inconsistent nap | Child may have sleep | Establish a consistent naptime |
| times of variable duration and | fragmentation | routine |
| low post-nap participation. | | Regulating duration of the |
| | | child's nap |
| | | Creating a relaxing nap |
| | | environment |
| | | Encourage physical activity |
| | | Avoiding stimulating activities |
| | | before bed |
| | underlying sleep disorder | Seeing a therapist |
| | Child may be experiencing | Seeing a therapist |
| | stress or anxiety | |

The social interactions among children are identified by analyzing their participation data and comparing them to each other. The duration of simultaneous participation of children within zones is measured. The duration of time each child interacts with other children is determined wherein the presence of the respective child with other children in each zone is interpreted as their interaction. The percentage of time each child interacts with other children is determined by dividing the duration of time the respective child interacts with each child by the total time the child participates in kindergarten activities. For each child, a plurality of social groups is determined with whom they spend time. These social groups are categorized based on the percentage of time the child interacts with members of each group: a high-interaction group (70% or more), a moderate-interaction group (50-70%), a casual-interaction group (30-50%), an occasional-interaction group (10-30%), and a low-interaction group (less than 10%).

To identify a child's leadership role, the count of times a child is the first to leave or enter a zone is determined and the number of children who follow that child out of or into the zone within a 3-minute is obtained. A ‘leadership score’ is then calculated by dividing the total number of children who follow that child by the number of times the child led the zone transition. Children who often pioneer zone changes are recognized as leaders. The child with the highest leadership score is identified as a leader, and those with scores exceeding half of this peak score are also identified as leaders.

The method evaluates the impact of zone-based kindergarten settings on children's development by analyzing participation data and over a period, e.g., six months, proposes reorganizations of zones and subdomains to enhance children's learning and overall development. By analyzing the participation data of individual children, the method determines the total time spent by children in each zone and subdomain. A participation percentage is obtained for each zone and subdomain. The participation percentage for each zone is calculated by dividing the total time spent by children in that zone by the total time spent by children in all zones. Similarly, the participation percentage for each subdomain is determined by dividing the total time spent by children in that particular subdomain by the total time spent by children in all subdomains. By comparing the participation percentages of zones and subdomains and their impact value on developed, underdeveloped, and overdeveloped criteria, the method identifies a plurality of redesign suggestions to reorganize kindergarten. These redesign suggestions aim to maintain the current participation percentage for zones and subdomains that influence the developed criteria while improving the participation percentage for zones and subdomains that impact the underdeveloped criteria. The redesign suggestions comprise rearranging the positions of zones, assigning a subdomain to its respective zone for a shorter duration, and assigning a subdomain to its respective zone for a longer duration.

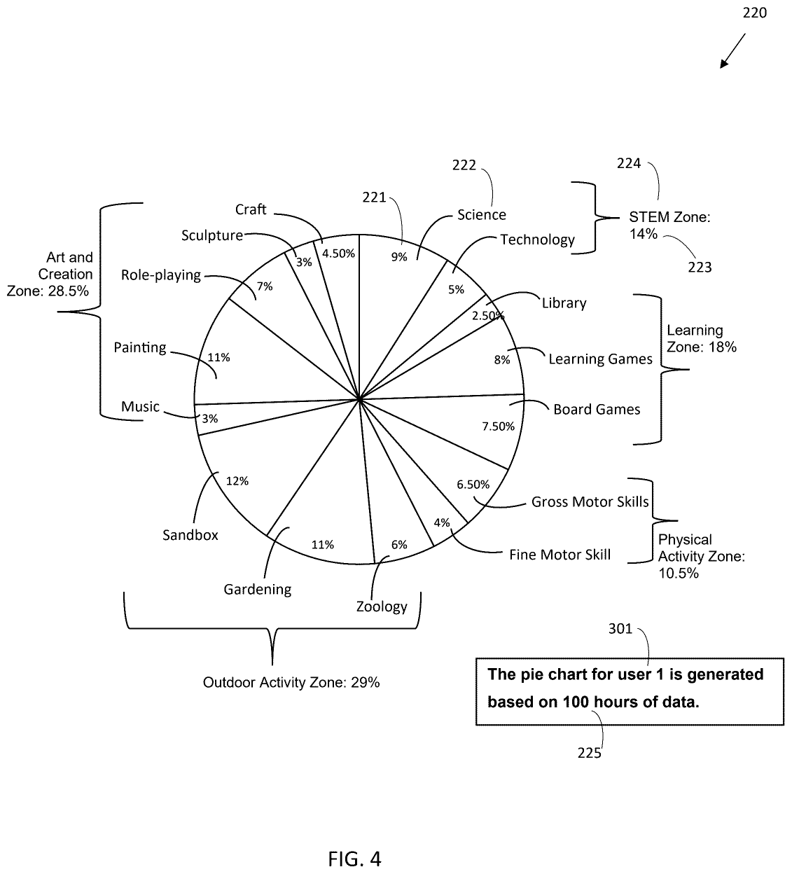

As shown in FIGS. 4 to 10, the report generation module generates a report 207 capturing the results of the assessment. The report comprises the time that each child spends in each zone and subdomain 220, the participation value of each child in interest criteria 230, the participation value of each child in development criteria 240, the impact values of zones and subdomains on the interest criteria 250, the impact values of zones and subdomains on the development criteria, a social interaction map for each child 260, and a dynamic characteristic map for each child 270.

Again, FIG. 4 shows a pie chart representing the time spent by a child (User 1) across different zones and subdomains 220. Each slice of the pie chart corresponds to a specific subdomain and contains a numerical value indicating the percentage of the child's participation in that subdomain. The numerical values allocated to the zones indicate the percentage of the child's participation in that zone. The total time of the child's participation in all the zones in a certain time is used to generate the pie chart. For instance, User 1 301 allocates 9% 221 of their total time (100 hours) 225 to the science subdomain 222 and 14% 223 to the STEM zone 224, indicating that during a 100-hour kindergarten experience, User 1 301 engages in activities associated with the science subdomains 222 for 9 hours and the STEM zone 224 for 14 hours.

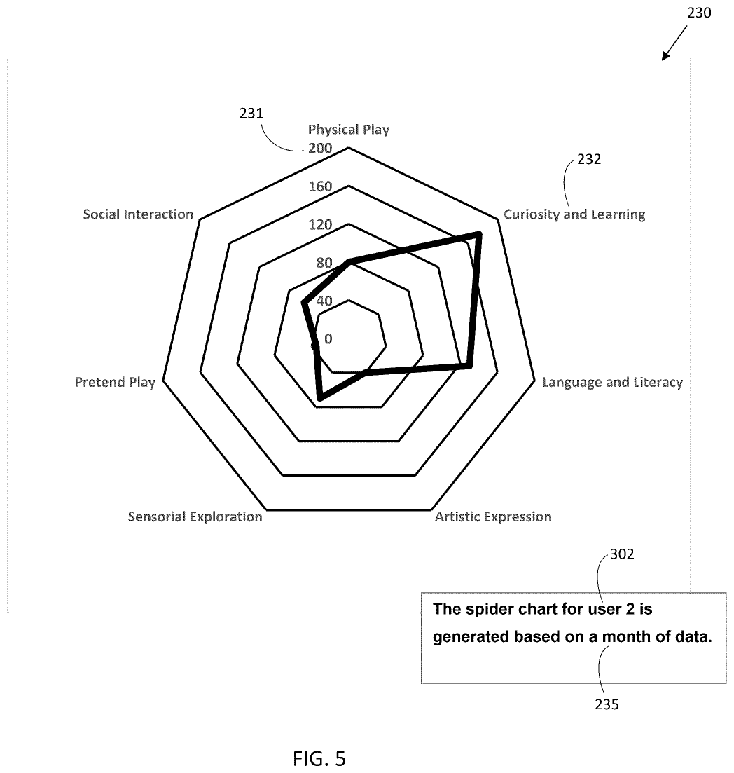

FIG. 5 illustrates the participation value of a child (User 2) in interest criteria through a spider chart 230. The participation value for each criterion indicates the level of participation of the child in that specific area. The participation values range from zero to a maximum value, which differs for each child and is determined by the duration of the analyzed data 235. The spider chart 230 is generated based on a month's worth of data, and for User 2 302, the maximum participation value 231 within that month is set at 200. Each interest criterion is represented by a node on the chart, and the participation value for that criterion is indicated at each node. These nodes are connected by lines, resulting in a pattern resembling a spider web. For instance, the participation value of User 2 302 in the curiosity and learning criterion 232 is 140, indicating a high level of participation in that particular area.

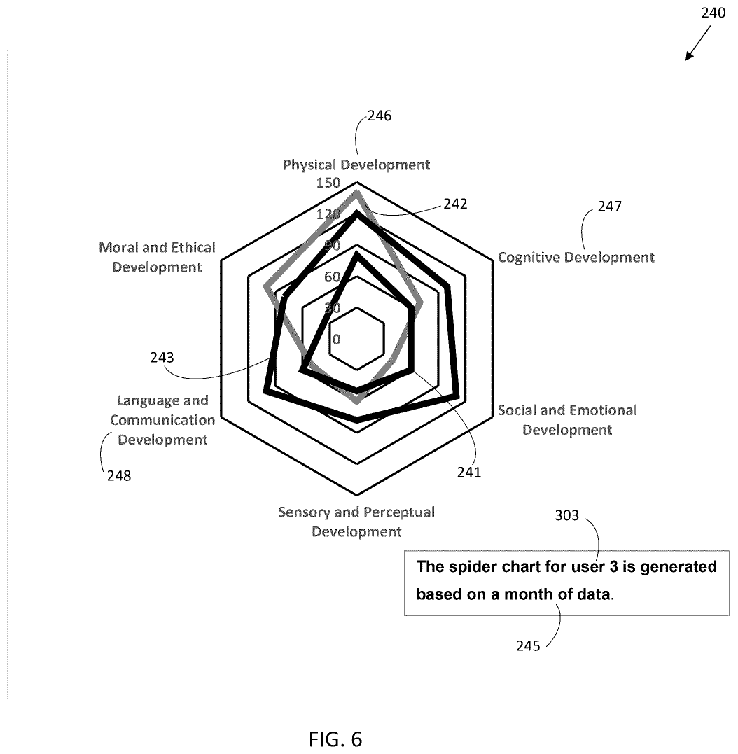

FIG. 6 illustrates the participation value of a child (User 3) in development criteria through a spider chart 240. The spider chart 240 consists of three spider webs: one representing the child's participation value in each development criterion 242, and two additional spider webs that establish the highest 243 and lowest 241 participation value requirements for the child. When a node indicating the participation value in criterion 242 exceeds the corresponding node representing the higher participation value requirement 243, it suggests that the child is overdeveloped in that criterion when a node falls between the nodes denoting the highest 243 and lowest 241 participation value requirements, it indicates that the child is developed in that criterion and when a node representing the participation value in criterion 242 is lower than the node representing the lower participation value requirement 241, it implies that the child is underdeveloped in that criterion. For instance, User 3 303 is overdeveloped in physical development 246, developed in cognitive development 247, and underdeveloped in language and communication development 248. The spider chart 240 is generated based on the duration of the analyzed data 245 which here is a month's worth of data.

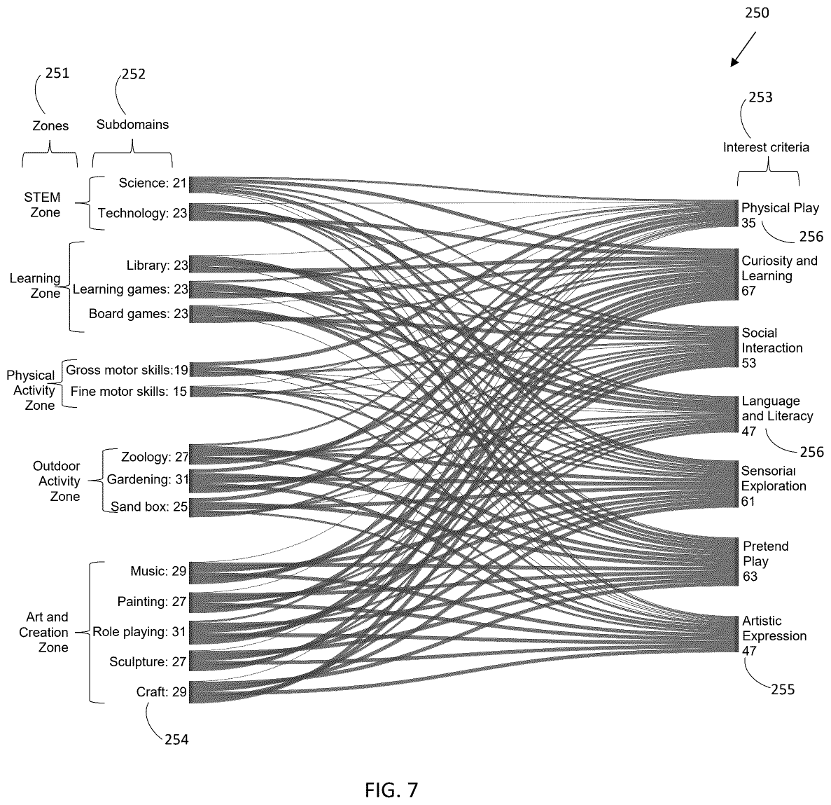

FIG. 7 displays the impact values of zones and subdomains on the interest criteria, presented in a snaky chart 250. This chart illustrates the zones 251, subdomains 252, interest criteria 253, and the impact of subdomains on each interest criterion. The lines indicate which interest criteria are influenced by the subdomains, with varying line thickness indicating the level of effectiveness (high effect with an impact value of 5, medium effect with an impact value of 3, low effect with an impact value of 1). The numbers associated with each subdomain represent the cumulative impact value it has on the criteria, while the numbers associated with each criterion represent the cumulative impact value it receives from the subdomains. For example, craft subdomain 254 demonstrates a high effect on artistic expression 255, a medium effect on language and literacy 256, and a low effect on physical play 257.

The report provides the impact values of zones and subdomains on the development criteria in the form of a snaky chart. The snaky chart captures the subdomains, development criteria, and the impact of subdomains on each development criterion.

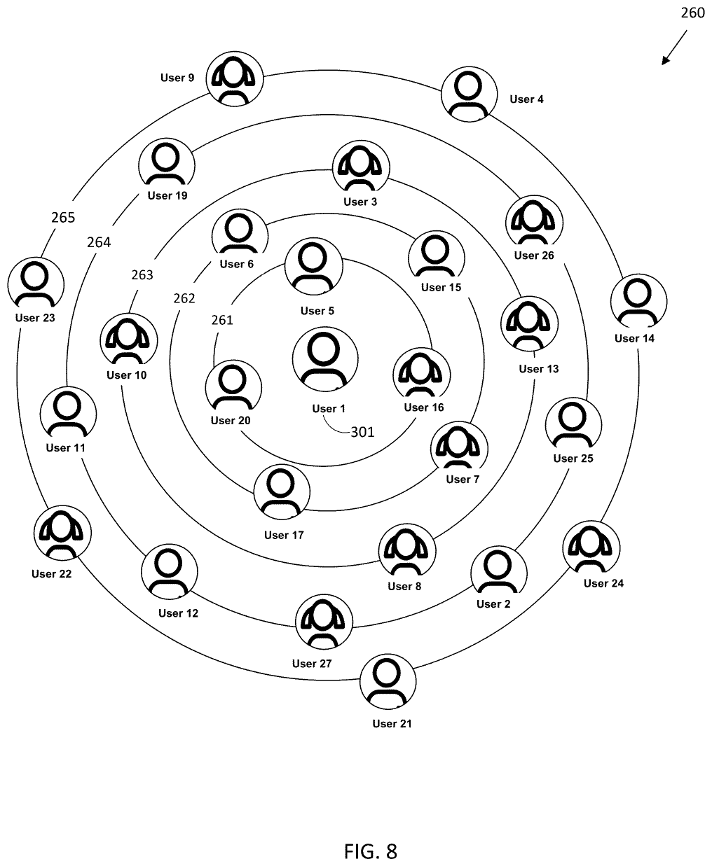

FIG. 8 presents a social interaction map for a child (User 1) in a kindergarten setting. The social interaction map 260 illustrates the child's interactions with other children in the kindergarten. The social interaction map 260 comprises a plurality of circles and on each circle pictures and names of children in kindergarten are located. Each circle represents a social group in which user 1 301 spends the same amount of time with its members. Social groups comprising a high-interaction group 261, in which the user spends 70% of their time or more with its members, a moderate-interaction group 262 in which the user spends 50 to 70% of their time with its members, a casual-interaction group 263 in which the user spends 30 to 50% their time with its members, an occasional-interaction group 264 in which the user spends 10 to 30% of their time with its members, and a low-interaction group 265 in which the user spends less than 10% of their time with its members.

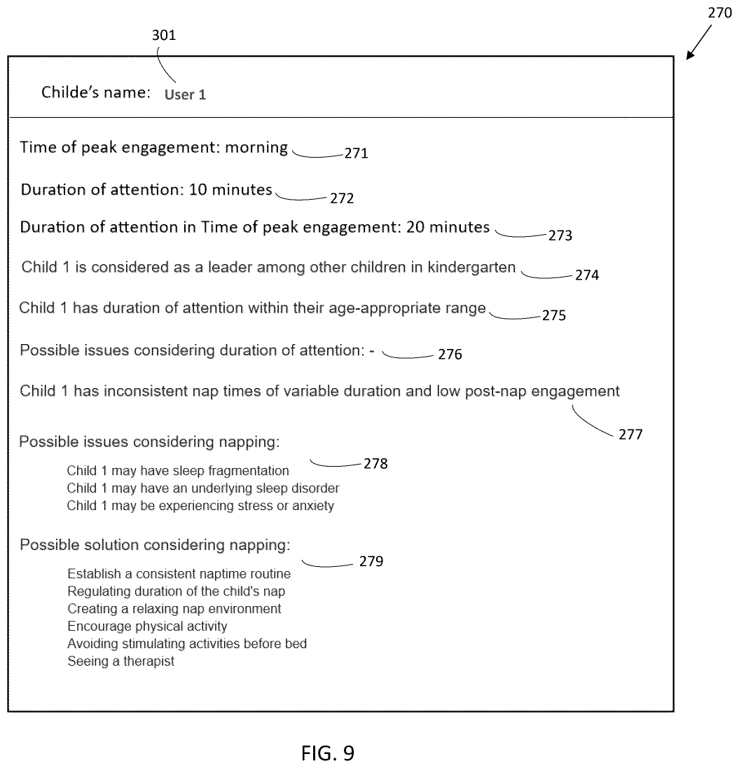

FIG. 9 illustrates a dynamic characteristic map 270 created for a child (user 1) 301, capturing various aspects of their behavior. This map comprises the child's time of peak engagement 271, total duration of attention 272, total duration of attention during the time of peak engagement 273, whether they exhibit leadership or follower characteristics 274, how their total duration of attention compares to the age-appropriate range 275, and their style of taking naps 277. The map addresses potential issues related to the child's total duration of attention 276 and style of taking naps 278 and provides possible solutions for addressing these issues 279. The dynamic characteristic map evolves as the child grows and develops.

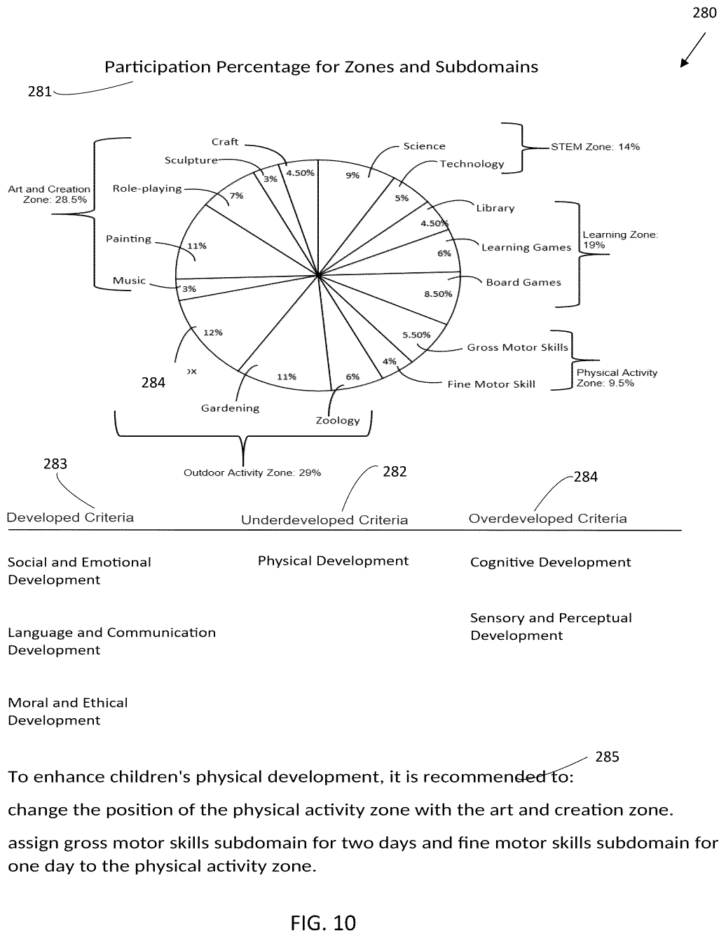

FIG. 10 illustrates an optimizing kindergarten setting map 280 comprising the participation percentage for zones and subdomains in the form of a pie chart 281, a table outlining the development criteria stages determining underdeveloped 282, developed 283, and overdeveloped criteria 284, and the redesign suggestions 285. The redesign suggests determining which zones should change their positions, and which subdomains should be assigned to their respective zone for a shorter or longer duration.

1. A method of collecting, analyzing, and assessing children's participation in activities within a kindergarten setting, comprising steps of:
a) dividing a kindergarten into a plurality of zones, wherein each zone has an RFID reader, wherein each of the plurality of zones representing a designated area focusing on distinct aspects of early childhood education and development;
b) assigning a set of activities to each zone, wherein a set of activities assigned to each of the plurality of zones is aligned with the corresponding aspect of early childhood education and development addressed within said zone;
c) obtaining data on each child's entrance and exit times for each zone from a data collector, wherein each child has an RFID tag;
d) generating zone attendance data by analyzing data obtained from the data collector, wherein zone attendance data indicates the time, frequency, and duration of each child's presence within each zone;
e) generating participation data for each child using zone attendance data, wherein participation data for each child indicates the time, frequency, duration, and sequence of the respective child's participation in the set of activities assigned to each zone, and wherein children's presence within each zone is interpreted as their participation in the set of activities assigned to the respective zone;
f) submitting participation data to a processor configured to assess children based on data indicative of their participation in the set of activities assigned to each of the plurality of zones;
g) identifying through the processor a plurality of factors related to children's characteristics comprising to their development level, interests and preferences, time of peak engagement, total duration of attention, napping patterns, social behaviors and interactions, potential strengths and problems in children's characteristics, and
h) generating reports on the plurality of factors related to children's characteristics and participation data.

2. The method of claim 1, wherein a rotation schedule is defined for the set of activities within each zone comprising steps of:
a) defining a plurality of main domains, wherein each main domain further comprises a plurality of subdomains and each subdomain further comprises a set of activities, and wherein the plurality of main domains cover a broad area of early childhood education and development and the plurality of subdomains break down each main domain into more specific areas, and the set of activities within each of the plurality of the subdomains address specific learning objectives of the corresponding subdomain;
b) assigning each of the plurality of the main domains to one of the plurality of zones;
c) selecting a subdomain from the plurality of subdomains associated with a corresponding main domain for a designated time within the zone;
d) providing the set of activities associated with the currently selected subdomain for children within the corresponding zone during the designated time, and
e) repeating steps c and d after the designated time has elapsed.

3. The method of claim 1, wherein identifying children's development level, comprising the steps of:
a) obtaining a plurality of developmental criteria;
b) determining the impact that each set of activities has on each criterion;
c) assigning a plurality of impact values to each set of activities, wherein the impact value represents the influence of the respective set of activities on the criterion;
d) establishing a plurality of participation values, wherein each of the plurality of the participation values determines each child's participation in each criterion, and wherein a participation value for a child in a criterion is established by multiplying the impact value that subdomains have on the respective criterion by the time the child spends in each subdomain relevant to that impact value and by summing the results of the multiplications;
e) obtaining a participation value requirement for each child in each criterion, and
f) categorizing individual children's development based on a comparison of the child's participation value (step d) and the participation value requirement (step e) in each criterion, into three stages, comprising:
f1) underdeveloped criterion, wherein the term implies that the participation value of the child in the respective criterion is lower than its participation value requirement;
f2) developed criterion, wherein the term implies that the participation value of the child in the respective criterion is equal to its participation value requirement, and
f3) overdeveloped criterion, wherein the term implies that the participation value of the child in the respective criterion is higher than its participation value requirement.

4. The method of claim 1, wherein
a) identifying each child's interests and preferences within the plurality of zones and subdomains, comprising the steps of:
a1) obtaining a mandatory participation time for children wherein the mandatory participation time refers to the time that children are directed to participate in activities by their teacher or other caregivers, and wherein the mandatory participation time is obtained through a user interface by the kindergarten teacher or other caregivers;
a2) generating participation time excluding mandatory participation time;
a3) determining zones and subdomains with the highest participation time excluding mandatory participation time for each child, as their interests and preferences;
b) identifying each child's interests and preferences within a plurality of interest criteria, comprising the steps of:
b1) obtaining a plurality of interest criteria wherein the interest criteria encapsulate a broad spectrum of children's interests;
b2) determining the impact that each subdomain has on each criterion;
b3) assigning a plurality of impact values to each subdomain, wherein the impact value represents the influence of the respective subdomain on the criterion;
b4) establishing a plurality of participation values, wherein each of the plurality of the participation values determines each child's participation in each interest criterion, and wherein a participation value for a child in an interest criterion is established by multiplying the impact value that subdomains have on the respective criterion by the time the child spends in each subdomain relevant to that impact value and by summing the results of the multiplications, and
b5) determining interest criterion with the highest participation value for each child, as their interests and preferences within the plurality of interest criteria.

5. The method of claim 1, wherein identifying children's time of peak engagement, comprising the steps of:
a) dividing the time spent by children in kindergarten into smaller segments, comprising morning, noon, afternoon, and periods before or after nap;
b) obtaining the frequency and duration of each child's participation in each zone during the time segments, and
c) identifying the time segment with the least frequency of zone changes and the longest duration of participation for each child, as their time of peak engagement.

6. The method of claim 1, wherein
a) identifying each child's total duration of attention, comprising the steps of:
a1) analyzing each child's participation data;
a2) computing the number of times each child enters each zone and the duration they spent at each entrance;
a3) calculating the duration of attention for each child in each zone by dividing the total duration of the child's presence in that zone by the number of times they enter the respective zone;
a4) identify the total duration of attention for each child by dividing the duration of attention for each child in each zone by the total number of zones;
b) identifying each child's total duration of attention during the child's time of peak engagement, comprising the steps of:
b1) analyzing each child's participation data during their time of peak engagement;
b2) computing the number of times each child enters each zone and the duration they spent at each entrance during their time of peak engagement;
b3) calculating the duration of attention for each child in each zone during their time of peak engagement by dividing the total duration of the child's presence in that zone by the number of times they enter the respective zone during their time of peak engagement, and
b4) identify the total duration of attention for each child during their time of peak engagement by dividing the duration of attention for each child in each zone during their time of peak engagement by the total number of zones during their time of peak engagement.

7. The method of claim 1, wherein identifying each child's napping patterns, comprising the steps of:
a) assigning a zone for the nap;
b) obtaining time and durations of each child's naps from their participation data, and
c) determining whether their nap times are consistent or inconsistent and whether their nap durations remain similar or vary across different napping sessions.

8. The method of claim 1, wherein identifying social interactions among children, comprising the steps of:
a) analyzing children's participation data and comparing them to each other;
b) measuring duration of simultaneous participation of children within zones;
c) determining, wherein the presence of the respective child with other children in each zone is interpreted as their interaction;
d) determining the percentage of time each child interacts with other children by dividing the duration of time the respective child interacts with each child by the total time the child participates in kindergarten activities;
e) determining a plurality of social groups for each child based on the percentage of time the child interacts with members of each group wherein the social groups comprising:
e1) a high-interaction group with 70% interaction or more among the group members;
e2) a moderate-interaction group with 50% to 70% interaction among the group members;
e3) a casual-interaction group with 30% to 50% interaction among the group members;
e4) an occasional-interaction group with 10% to 30% interaction among the group members, and
e5) a low-interaction group with less than 10% interaction among the group members.

9. The method of claim 1, wherein identifying each child's leadership role, comprising the steps of:
a) determining the count of times a child is the first to leave or enter a zone;
b) obtaining the number of children who follow the respective child out of or into the zone within a 3-minute;
c) calculating a leadership score by dividing the total number of children who follow the respective child by the number of times the child led the entrance and exit zones, and
d) identifying the child with the highest leadership score and children with leadership score exceeding half of the highest leadership score as leaders.

10. The method of claim 1, wherein
a) Identifying potential problems and strengths in children's total duration of attention, comprising the steps of:
a1) establishing a range of age-appropriate duration of attention for each child;
a2) comparing children's total duration of attention with their range of age-appropriate duration of attention;
a3) categorizing children based on the comparison in step b into three groups comprising:
i) children with total duration of attention shorter than their age-appropriate range;
ii) children with total duration of attention longer than their age-appropriate range,
iii) children with total duration of attention within their age-appropriate range, and
wherein children are categorized into groups i and ii could indicate potential problems and strengths;
b) Identifying potential problems and strengths in children's napping patterns, comprising the steps of:
b1) identifying each child's total post-nap duration of attention;
b2) identifying each child's total pre-nap duration of attention;
b3) determining whether each child exhibits high or low post-nap participation, wherein high post-nap participation indicates a longer total post-nap duration of attention compared to total pre-nap duration of attention and total duration of attention and wherein low post-nap participation indicates a shorter total post-nap duration of attention compared to their total pre-nap duration of attention and total duration of attention;
b4) categorizing children into eight groups comprising:
i) children with consistent nap times of similar duration and high post-nap participation;
ii) children with consistent nap times of variable duration and high post-nap participation;
iii) children with inconsistent nap times of similar duration and high post-nap participation;
iv) children with inconsistent nap times of variable duration and high post-nap participation;
v) children with consistent nap times of similar duration and low post-nap participation;
vi) children with consistent nap times of variable duration and low post-nap participation;
vii) children with inconsistent nap times of similar duration and low post-nap participation;
viii) children with inconsistent nap times of variable duration and low post-nap participation, and
wherein children are categorized into groups could indicate potential problems and strengths.

11. The method of claim 1, wherein the method further comprises the step of evaluating the impact of zone-based kindergarten settings and proposing redesign suggestions using the participation data, comprising the steps of:
a) determining total time spent by children in each of the plurality of zones and total time spent by children in each of the plurality of subdomains within a zone;
b) calculating participation percentages for each of the plurality of zones and each of the plurality of subdomains, wherein participation percentages for each zone are calculated by dividing the total time spent in the respective zone by the total time spent in all zones and for each subdomain are calculated by dividing the total time spent in the respective subdomain by the total time spent in all subdomains;
c) identifying a plurality of redesign suggestions for zone and subdomain organization based on the participation percentages of zones and subdomains and their impact value on developed, underdeveloped, and overdeveloped criteria, wherein the redesign suggestions aim to maintain participation percentages for zones and subdomains impacting developed criteria and improve participation percentages for zones and subdomains impacting underdeveloped criteria;
d) generating redesign suggestions comprising at least one of the following:
d1) rearranging the positions of zones within the kindergarten setting;
d2) adjusting the designated time for a subdomain within its respective zone for a shorter duration, and
d3) adjusting the designated time for a subdomain within its respective zone for a longer duration.

12. The method of claim 1, wherein the report comprises a plurality of charts for each child determining the time that each child spends in each zone and subdomain, the participation value of each child in interest criteria, the participation value of each child in development criteria, a social interaction map for each child, and a dynamic characteristic map for each child and a chart determining the impact values of zones and subdomains on the interest criteria, a chart determining the impact values of zones and subdomains on the development criteria, and an optimizing kindergarten setting map.

13. The method of claim 1, wherein the optimizing kindergarten setting map comprising the participation percentages for zones and subdomains, developed, underdeveloped, and overdeveloped criteria, and redesign suggestions.
